(19) United States
(12) Patent Application Publication
(10) Pub. No.: US 2004/0132689 A1
(43) Pub. Date: Jul. 8, 2004

(54) Aqueous preparation containing a shark-derived chondroitin iron sulfate colloid

(76) Inventors: Seiji Nishida, Osaka-shi (JP); Naohisa Katayama, Osaka-shi (JP); Takashi Katsumata, Osaka-shi (JP); Makoto Sato, Osaka-shi (JP)

Correspondence Address: KUBOVCIK & KUBOVCIK, SUITE 710, 900 17TH STREET NW, WASHINGTON, DC, 20006

(21) Appl. No.: 10/740,565

(22) Filed: Dec. 22, 2003

(30) Foreign Application Priority Data
Dec. 27, 2002 (JP) 2002-381167

(51) Int. Cl. A61K 31/737; C08B 37/00

(52) U.S. Cl. 514/54; 536/54

(57) ABSTRACT

A stabilized aqueous preparation including a shark-derived chondroitin iron sulfate colloid and a trace element. An aqueous preparation of the present invention is pharmaceutically stable, is stable in an infusion such as a high-calorie intravenous nutrition infusion, and can be safely applied in clinical use without worry of bovine spongiform encephalopathy (BSE) infection.

### TECHNICAL FIELD OF THE INVENTION

The present invention relates to an aqueous preparation containing essential trace elements for high-calorie intravenous nutrition infusion which is capable of preventing and treating various deficiency symptoms derived from a lack of essential trace elements. More specifically, the present invention relates to an aqueous preparation for high-calorie intravenous nutrition infusion having excellent pharmaceutical safety and pharmaceutical stability containing essential trace elements such as iron, selenium, zinc, copper, iodine, and/or manganese, used for infusion therapy.

### BACKGROUND OF THE INVENTION

Conventionally, high-calorie intravenous nutrition infusion therapy is typically performed and is widely used for a patient who has no or insufficient oral or enteral feeding ability. In high-calorie intravenous nutrition infusion therapy, generally, carbohydrates or the like as a source of energy, amino acids as a source of protein, and other components such as electrolytes and vitamins are intravenously administered. However, when a high-calorie intravenous nutrition infusion containing the above components is used for a long period of time, deficiency symptoms accompanied by a lack of essential trace elements for humans, namely, iron, selenium, zinc, copper, iodine, manganese, molybdenum, and chromium, develop.

When iron is parenterally supplied, there is a problem in terms of toxicity because an ionic iron compound binds to transferrin under a saturation condition and also binds to a plasma protein, causing shock or the like. Thus, there is a need to devise a means of supplying iron in a colloidal form with few side effects. As iron ion to be parenterally supplied to humans, ferric chloride is generally used. In an aqueous solution, such ferric chloride exists as a ferric hydroxide colloid particle. Such a colloid particle includes oxy chloride (FeOCl) in addition to ferric oxide (Fe₂O₃) and water; is a hydrophobic colloid positively charged by dissociation to FeO⁺ and Cl⁻; and has a tendency to aggregate. If the pH value thereof rises to about 3 or more, it will precipitate as a result of such aggregation (See, for example, “Colloid Chemistry”, written by B. Jirgensons et al., and translated under the editorship of Bunichi Tamamushi, Baifukan, Tokyo, 1967).

In Japan, as a protective colloid, an iron colloid solution containing a chondroitin sulfate having few side effects and high iron utilization ratio has been used. For example, chondroitin iron sulfate colloid is commercially available as an intravenous injection preparation for iron deficiency anemia under the name Blutal (trade name, Dainippon Pharmaceutical Co., Ltd.). In addition, a preparation containing chondroitin iron sulfate colloid as a supplement of essential trace elements for high-calorie intravenous nutrition is also commercially available as Elemenmic injection and Elemate injection (trade names, Ajinomoto Pharma, Co., Ltd.), Mineralin injection and Parmirin injection (trade names, Nippon Pharmaceutical Co., Ltd./Takeda Chemical Industries, Ltd.), and the like.

Each of these commercially available preparations containing chondroitin iron sulfate colloid is prepared using a bovine-derived sodium chondroitin sulfate as a protective colloid. In 1996, however, it was announced in Great Britain that there was a causal relationship between the onset in a patient of Variant Creutzfeldt-Jakob disease (vCJD) and mad cow disease, i.e., bovine spongiform encephalopathy (BSE). An abnormal prion protein regarded as a cause of BSE is heat-stable. Therefore, it is considered that a high-temperature, high-pressure treatment and an alkali treatment will not perfectly inactivate the prion protein.

In Japan, in consideration of the outbreak of bovine BSE in Europe, for any drug or the like manufactured using a raw material derived from cows or the like, there is a need that manufacturers and so on take measures for ensuring quality and safety. Accordingly, a medicine security chief of the Ministry of Health, Labour, and Welfare published a bureau notice No. 1226, “For ensuring qualities and safeties of drugs and so on manufactured using a bovine-derived product or the like as a raw material” dated Dec. 12, 2000, requiring guided self inspection, preparation of written acknowledgement, and so on by drug manufacturers or the like.

Bovine-derived chondroitin sulfate has been isolated and purified from bovine tracheae. However, there is a demand to use a safer chondroitin sulfate replacing a bovine-derived product.

An object of the present invention is to provide an aqueous preparation containing a shark-derived chondroitin iron sulfate colloid not containing any BSE causative agent, which is safe and excellent in pharmaceutical stability, and additionally is stable when mixed in an infusion such as a high-calorie intravenous nutrition infusion.

More specifically, in the present invention, an aqueous preparation containing a shark-derived chondroitin iron sulfate colloid prepared using, instead of a bovine-derived sodium chondroitin sulfate, a shark-derived sodium chondroitin sulfate not containing any BSE causative agent and which is safe, inhibits the decline of a pH value after sterilization and improves heat stability.

### SUMMARY OF THE INVENTION

The inventors of the present invention have carefully considered various ways of obtaining an aqueous preparation not containing any BSE causative agent, which is safe and excellent in pharmaceutical stability, and additionally is stable when mixed in an infusion such as a high-calorie intravenous nutrition infusion. As a result, the inventors of the present invention have found that an aqueous preparation containing a shark-derived chondroitin iron sulfate colloid in which shark-derived sodium chondroitin sulfate is provided as a protective colloid and a pH buffer inhibits the decline of pH after sterilization and is excellent in stability. By devoting themselves to intensive research relating to these findings, the present invention has finally been completed.

That is, the present invention relates to:

(1) a stabilized aqueous preparation comprising a shark-derived chondroitin iron sulfate colloid and a trace element,

(2) an aqueous preparation as described in item (1) above, further comprising a pH buffer,

(3) an aqueous preparation as described in item (1) or (2) above, wherein the shark-derived chondroitin iron sulfate colloid is produced from a shark-derived chondroitin sulfate,

(4) an aqueous preparation as described in item (3) above, wherein the shark-derived chondroitin sulfate is an alkali metal salt of the shark-derived chondroitin sulfate,

(5) an aqueous preparation as described in item (3) above, wherein the shark-derived chondroitin sulfate is shark-derived sodium chondroitin sulfate,

(6) an aqueous preparation as described in item (5) above, wherein the shark-derived sodium chondroitin sulfate has an average molecular weight of from about 10,000 to about 25,000; a limiting viscosity of from about 0.27 to about 0.65 dl/g; and a sulfur content of from about 6.4 to about 7.0 wt/wt %,

(7) an aqueous preparation as described in item (2) above, wherein the pH buffer is one or more compounds selected from the group consisting of glycine, L-valine, L-arginine, L-histidine, acetic acid, malonic acid, maleic acid, succinic acid, malic acid, tartaric acid, citric acid, L-sodium glutamate, disodium phthalate, and disodium fumarate,

(8) an aqueous preparation as described in item (7) above, wherein the pH buffer is glycine, malic acid, citric acid or L-sodium glutamate,

(9) an aqueous preparation as described in item (7) above, wherein the trace element is one or more elements selected from the group consisting of selenium, zinc, copper, iodine, and manganese,

(10) an aqueous preparation as described in item (9) above, wherein the trace elements are zinc, copper, and iodine,

(11) an aqueous preparation as described in item (9) above, wherein the trace elements are zinc, copper, iodine, and manganese,

(12) an aqueous preparation as described in item (9) above, wherein the trace elements are selenium, zinc, copper, iodine, and manganese,

(13) an aqueous preparation as described in item (1) or (2) above, wherein a weight ratio of iron: shark-derived sodium chondroitin iron sulfate colloid is from about 1:4 to about 1:6,

(14) an aqueous preparation as described in item (13) above, wherein the weight ratio of iron : shark-derived chondroitin iron sulfate colloid is about 1:5,

(15) an aqueous preparation as described in item (2) above, wherein the concentration of the pH buffer is from about 0.01 to about 1.0 wt/vol %,

(16) an aqueous preparation as described in item (1) or (2) above, wherein the preparation contains a shark-derived chondroitin iron sulfate colloid produced by adding an aqueous ferric salt solution and an aqueous alkali metal hydroxide solution to an aqueous shark-derived chondroitin sulfate solution so that the pH of the resulting mixture is adjusted to a pH within a range of from about 5.5 to about 7.5,

(17) a method for manufacturing an aqueous preparation containing a shark-derived chondroitin iron sulfate colloid, comprising:

adding an aqueous ferric salt solution and an aqueous alkali metal hydroxide solution to an aqueous shark-derived chondroitin sulfate solution so that the pH of the resulting mixture is adjusted to a pH within a range of from about 5.5 to about 7.5; heating the resulting mixture; and adding a pH buffer and a trace element; and

(18) a method for manufacturing an aqueous preparation containing a shark-derived chondroitin iron sulfate colloid as described in item (17) above, wherein the shark-derived chondroitin sulfate is shark-derived sodium chondroitin sulfate, and the alkali metal hydroxide is sodium hydroxide.

### DETAILED DESCRIPTION OF THE INVENTION

An aqueous preparation of the present invention, which includes a shark-derived chondroitin iron sulfate colloid, a trace element, and a pH buffer (hereinafter sometimes referred to as the aqueous preparation of the present invention), can be manufactured by adding an aqueous ferric salt solution and an aqueous alkali metal hydroxide solution to an aqueous shark-derived chondroitin sulfate solution so that the pH of the resulting mixture is adjusted to a pH within the range of from about 5.5 to about 7.5; heating the resulting mixture; and adding a pH buffer and a trace element.

The shark-derived chondroitin sulfate is, for example, an alkali metal salt such as a sodium salt or potassium salt of a shark-derived chondroitin sulfate and, preferably, is shark-derived sodium chondroitin sulfate. The shark-derived sodium chondroitin sulfate may be, for example, one which is derived from shark cartilage and has such physical properties that an average molecular weight is from about 10,000 to about 25,000; a limiting viscosity is from about 0.27 to about 0.65 dl/g (measured by capillary tube viscometer); and a sulfur content is from about 6.4 to about 7.0 wt/wt %. Preferably, the shark-derived sodium chondroitin sulfate is one in which a composition ratio of chondroitin-4-sulfate (chondroitin sulfate A) : chondroitin-6-sulfate (chondroitin sulfate C) is about 1:3.

The chondroitin sulfate is a linear polymeric polysaccharide having a repetitive structure with disaccharide units of [→4-glucuronic acid β1→3N-acetyl-D-galactosamine β1→] and is a poly anion having a high negative charge in which isomers are present depending on the number of sulfate groups bound to such disaccharide units and the binding positions thereof. Table 1 shows a comparison of each isomer-composition ratio of sodium chondroitin sulfate derived from shark cartilage and from bovine tracheae (an average molecular weight of 20,000 to 25,000).

TABLE 1

Isomer composition ratio

| | Shark-cartilage- | Bovine-trachea- |
|---|---|---|
| Position | derived Chs | derived Chs |
| of sulfate | Molecular weight | Molecular weight |
| group | 20,000-25,000 | 15,000-20,000 |
| ΔDi-0S | 4.4% | 5.1% |
| ΔDi-4S | 21.0% | 47.5% |
| ΔDi-6S | 60.4% | 43.0% |
| ΔDi—diS_{D} | 12.1% | 1.1% |
| ΔDi—diS_{E} | 2.0% | 0.7% |

(Note)
Chs: sodium chondroitin sulfate.
ΔDi-0S: A sulfate group is not bonded.
ΔDi-4S: A sulfate group is bonded to the C-4 position of N-acetyl-D-galactosamine.
ΔDi-6S: A sulfate group is bonded to the C-6 position of N-acetyl-D-galactosamine.
ΔDi-diS_{D}: A sulfate group is bonded to the C-6 position of N-acetyl-D-galactosamine, and a sulfate group is bonded to the C-2 position of D-glucuronic acid.
ΔDi-diS_{E}: Sulfate groups are bonded to the C-4 and C-6 positions of N-acetyl-D-galactosamine.

Examples of the ferric salt include a compound containing ferric iron which can be used in the body such as ferric chloride hexahydrate (FeCl₃.6H₂O), ferric citrate (FeC₆H₅O₇), iron oxyhydroxide (FeO(OH)), ferric nitrate (Fe(NO₃)₃.9H₂O), iron oxide (Fe₂O₃), iron sulfate (Fe₂(SO₄)₃.nH₂O), or iron phosphate (FePO₄.nH₂O). Ferric salt changes to ferric hydroxide in an aqueous solution, and the shark-derived chondroitin sulfate is used as a protective colloid of the hydrophobic colloid solution of the ferric hydroxide. Of these, a ferric chloride such as ferric chloride hexahydrate (FeCl₃.6H₂O) is preferable. The weight ratio of iron:shark-derived sodium chondroitin sulfate is in the range of about 1:4 to about 1:6, and is preferably about 1:5.

Examples of the alkali metal hydroxide include sodium hydroxide or potassium hydroxide, preferably sodium hydroxide.

The pH buffer to be used may be any compound having a function of buffering the pH value. Preferred buffers are glycine, L-valine, L-arginine, L-histidine, acetic acid, malonic acid, maleic acid, succinic acid, malic acid, tartaric acid, citric acid, L-sodium glutamate, disodium phthalate, and disodium fumarate. One or two or more of these compounds may be selected and used.

Examples of the trace elements include selenium, zinc, copper, iodine, and manganese. These metal elements usually exist in the preparation in ionic form. Preferably, the trace element comprises 3 elements of zinc, copper and iodine, 4 elements of zinc, copper, manganese and iodine, or 5 elements of zinc, copper, manganese, selenium and iodine.

Selenium used as the trace element includes compounds such as selenious acid, sodium selenite, and sodium selenate. Zinc salts such as sulfates and chlorides are used, and examples thereof include zinc sulfate heptahydrate and zinc chloride. Copper salts such as sulfates and chlorides are used, and examples thereof include copper sulfate pentahydrate, cuprous chloride, and cupric chloride. Iodine to be used includes compounds such as potassium iodide and sodium iodide. Manganese salts such as sulfates and chlorides are used, and examples thereof include manganese sulfate pentahydrate and manganese chloride tetrahydrate.

More specifically, the aqueous preparation of the present invention can be manufactured by adding an aqueous ferric salt solution and an aqueous alkali metal hydroxide solution to an aqueous shark-derived chondroitin sulfate solution so that the pH of the resulting mixture is adjusted to a pH within the range of from about 5.5 to about 7.5 and chondroitin iron sulfate colloid is formed; heating the mixture containing the chondroitin iron sulfate colloid; adding a pH buffer to the mixture after heating; adding one or two or more of a selenium compound, zinc salt, copper salt, iodide, and manganese salt; adjusting the pH to about 5.8; and sterilizing the resultant under high-pressure steam. The temperature of heating is preferably from about 100° C. to 121° C. The time of heating is preferably from about 10 to 30 minutes.

In the manufacturing process of the present invention, an aqueous preparation containing a shark-derived chondroitin iron sulfate colloid and trace elements obtained by adding a base such as alkali metal hydroxide (preferably sodium hydroxide) as is or as an aqueous solution without an addition of a pH buffer to adjust the pH of the final aqueous preparation to about 5.8 after the addition of trace element compounds, can be used in the same way as the other preparation of the present invention.

In the process of adding the trace elements, it is preferred that the solution is controlled so that the pH is from about 5.3 to 6.3, preferably from about 5.6 to 6.0, more preferably about 5.8, after one or more compounds selected from the group consisting of a selenium compound, zinc salt, copper salt, iodide and manganese salt are added to the aqueous solution comprising a shark-derived chondroitin iron sulfate colloid. Then iodide is added to the solution.

In the manufacturing method of the present invention, an appropriate amount of the solution prepared by dissolving the ferric salt (e.g., ferric chloride hexahydrate) in a water for injection and an appropriate amount of the aqueous solution of alkali metal hydroxide (e.g., sodium hydroxide) are added with stirring to the solution prepared by dissolving shark-derived chondroitin sulfate (e.g., sodium chondroitin sulfate) in a water for injection, the amount of the shark-derived chondroitin sulfate corresponding to the above weight ratio of shark-derived sodium chondroitin sulfate to iron. It is preferable to adjust the pH of the mixture to any constant pH within the range of from about 5.5 to about 7.5.

The concentration of the aqueous ferric salt (e.g., ferric chloride hexahydrate) solution to be added is generally in the range of from about 3 to about 62 wt/vol %, preferably about 13 to about 32 wt/vol %.

The concentration of the aqueous alkali metal hydroxide (e.g., sodium hydroxide) solution to be added is generally in the range of from about 1 to about 28 wt/vol %, preferably from about 2 to about 7 wt/vol %.

The concentration of the aqueous chondroitin sulfate solution is generally in the range of from about 3 to about 30 wt/vol %, preferably about 4 to about 20 wt/vol %.

The concentration of the pH buffer to be used is generally about 1 wt/vol % or less, preferably from about 0.01 to about 1 wt/vol %.

The mixture containing the ferric salt, the alkali metal hydroxide, and the chondroitin sulfate is stirred sufficiently to maintain the pH of the mixture to a pH within the range of 5.5 to about 7.5 and to form the chondroitin iron sulfate colloid.

The time of reaction to form the chondroitin iron sulfate colloid may be appropriately determined by a person skilled in the art. In general, it may be in the range of from about an hour to about 6 hours. The temperature of the reaction may be appropriately selected by a person skilled in the art. Preferably, it is in the range of about 5° C. to about 25° C.

The thus-obtained aqueous preparation of the present invention may be used as an injection after sterilization, if required. In addition, separate containers can be filled with the solution in small portions (e.g., 1, 2, or 4 mL each), sealed, and subjected to sterilization (e.g., high-pressure steam sterilization). Desirably, the aqueous preparation of the present invention has a pH in the range of from about 4.5 to about 6.0.

As the container to encapsulate the aqueous preparation of the present invention, for example, a glass container (such as an ampoule), and a container made of a plastic material such as polypropylene, including a pre-filled type syringe, may be used.

The injection of the present invention can be administered to humans safely, while scarcely causing any side effects, in accordance with methods known per se. The amount of elements contained in the aqueous preparation of the present invention to be administered is, as a daily dose per adult person, from about 0.9 to about 720 μmol of iron, from about 0.025 to about 5.0 μmol of selenium, from about 3.85 to about 210 μmol of zinc, from about 0.9 to about 55 μmol of copper, from 0 to about 11 μmol of iodine, and from 0 to about 51 μmol of manganese; preferably from about 9 to about 720 μmol of iron, from about 0.25 to about 2.5 μmol of selenium, from about 38.5 to about 61.5 μmol of zinc, from about 9.1 to about 27.3 μmol of copper, from about 0.6 to about 1.1 μmol of iodine, and from 0 to about 14.5 μmol of manganese. It is desirable that these elements are contained in from 2 to 20 mL of the aqueous solution. The aqueous preparation of the present invention may optionally include an additional element such as chromium, molybdenum, cobalt, and fluorine.

### EXAMPLES

Hereinafter, the present invention will be described more specifically with reference to examples.

### Example 1

An aqueous ferric chloride solution obtained by dissolving 94.6 g of ferric chloride into 390 mL of purified water were fed into a solution of shark chondroitin sulfate obtained by dissolving 97.74 g of shark-cartilage-derived sodium chondroitin sulfate (molecular weight of about 10,000) into 1480 mL of purified water, in a 5-fold amount of the shark-cartilage-derived sodium chondroitin sulfate to the amount of iron (Fe) in weight ratio, with stirring, while adding an amount of an aqueous sodium hydroxide solution necessary to maintain the pH at about 6.5, followed by purified water to adjust to a volume of 5000 mL. Subsequently, the resultant mixture was heated at 110° C. for 20 minutes to prepare chondroitin iron sulfate colloid solution with 4 mg/mL of iron concentration. These operations were repeated once more and the total volume of the obtained chondroitin iron sulfate colloid solution was 10 L.

172.50 g of zinc sulfate were dissolved in 500 mL of purified water to prepare a zinc sulfate solution. Also, 12.48 g of copper sulfate and 1.66 g of potassium iodide were each dissolved in 250 mL of purified water to prepare a copper sulfate solution and a potassium iodide solution.

To 100 mL portions of the chondroitin iron sulfate colloid solution, an appropriate amount of purified water was added, and a pH buffer corresponding to respective concentrations as described below was added. If necessary, after adjusting the pH of each mixture to about 5.8, 10 mL of the zinc sulfate solution and 5 mL of the copper sulfate solution were added in order, and the pH was adjusted to 5.8 by adding an aqueous sodium hydroxide solution (1 wt/vol %). Further, 5 mL of the potassium iodide solution was added, and purified water was added to obtain a solutions that are each 400 mL in total volume.

Each solution was filtered using a membrane filter with a pore size of 0.22 μm, and 2 mL of the filtrate was filled into each of 20 barium-free colorless ampoules, followed by melt sealing. Subsequently, the ampoules were subjected to a high-pressure steam sterilization to prepare respective aqueous preparations containing various buffers.

In the samples, pH buffers of glycine, L-valine, L-arginine, L-histidine, acetic acid, malonic acid, maleic acid, succinic acid, malic acid, tartaric acid, citric acid, sodium glutamate, disodium phthalate, and disodium fumarate were used in amounts so as to adjust the concentrations to 0, 0.01, 0.05, 0.1, 0.2, and 1.0 wt/vol %.

For each of the samples, the pH before and after the high-pressure steam sterilization was measured, and a buffer capacity (%), an indication of buffer action, was calculated using the following equation and evaluated. The results are shown in Table 2.

Buffer capacity (%)=(difference of pH before and after sterilization of sample without buffer−difference of pH before and after sterilization of sample with buffer)/(difference of pH before and after sterilization of sample without buffer)

TABLE 2
Test results of the buffer capacity (%)
calculated from the pH before and after
the high-pressure steam sterilization of
samples added with various buffers

| Buffer added to | Concentration of buffer in sample (wt/vol %) | | | | |
|---|---|---|---|---|---|
| sample | 0.01 | 0.05 | 0.1 | 0.2 | 1.0 |
| Glycine | 11.3 | 40.8 | 59.2 | 74.6 | 91.5 |
| L-valine | −1.5 | 15.4 | 36.9 | 49.2 | 90.8 |
| L-arginine | 3.2 | 20.6 | 39.7 | 50.8 | 93.7 |
| L-histidine | 0 | 22.4 | 58.2 | 80.6 | 100.0 |
| Acetic acid | 4.2 | 21.1 | 36.6 | 50.7 | 84.5 |
| Malonic acid | 6.7 | 21.3 | 36.0 | 50.7 | 104.0 |
| Maleic acid | 11.3 | 28.2 | 52.1 | 66.2 | 91.5 |
| Succinic acid | 12.9 | 30.0 | 44.3 | 65.7 | 87.1 |
| Malic acid | 10.6 | 15.2 | 18.2 | 33.3 | 119.7 |
| Tartaric acid | 10.1 | 30.4 | 36.2 | 49.3 | 95.7 |
| Citric acid | 18.1 | 16.7 | 12.5 | 63.9 | 98.6 |
| Disodium phthalate | 4.3 | 18.8 | 24.6 | 39.1 | 76.8 |
| L-sodium glutamate | 7.4 | 17.6 | 41.2 | 51.5 | 88.2 |
| Disodium fumarate | 1.4 | 8.2 | 11.0 | 16.4 | — |

(Note)
Each value represents buffer capacity (%).

As shown in Table 2, buffer capacity was dependent on concentration in the range of from 0.01 wt/vol % to 1.0 wt/vol % for each of the buffers except for buffer concentrations of 0.01 wt/vol % of L-valine, 0.01 wt/vol % of L-histidine, and 1.0 wt/vol % of disodium fumarate.

### Example 2

An aqueous ferric chloride solution obtained by dissolving 94.6 g of ferric chloride into 390 mL of purified water were fed into a solution of shark chondroitin sulfate obtained by dissolving 97.74 g of shark-cartilage-derived sodium chondroitin sulfate (molecular weight of about 10,000) into 1480 mL of purified water, in a 5-fold amount of the shark-cartilage-derived sodium chondroitin sulfate to the amount of iron (Fe) in weight ratio, with stirring, while adding an amount of an aqueous sodium hydroxide solution necessary to maintain the pH at about 6.5, followed by purified water to adjust to a volume of 5000 mL. Subsequently, the resultant mixture was heated at 110° C. for 20 minutes to prepare chondroitin iron sulfate colloid solution with 4 mg/mL of iron concentration.

8.625 g of zinc sulfate was dissolved in 100 mL of purified water to prepare a zinc sulfate solution. Also, 0.624 g of copper sulfate and 0.083 g of potassium iodide were each dissolved in 25 mL of purified water to prepare a copper sulfate solution and a potassium iodide solution.

To 250 mL portions of the chondroitin iron sulfate colloid solution, an appropriate amount of purified water was added, and a pH buffer of malic acid or citric acid corresponding to a concentration of 0.2 wt/vol % was added. After adjusting the pH to about 5.8, the zinc sulfate solution and the copper sulfate solution were added in order, and the pH value was adjusted to 5.8 by adding an aqueous sodium hydroxide solution (1 wt/vol %). Further, the potassium iodide solution was added, and purified water was added to obtain solutions of 1,000 mL in total volume each.

Each solution was filtered using a membrane filter with a pore size of 0.22 μm, and 2 mL of the filtrate was filled into each of 215 barium-free colorless ampoules, followed by melt sealing. Subsequently, the ampoules were subjected to a high-pressure steam sterilization to prepare aqueous preparations containing respective buffers of malic acid or citric acid.

For the obtained samples, stability evaluation at 70° C. storage was conducted. The results are shown in Table 3.

TABLE 3

| | | | | 70° C. | | |
|---|---|---|---|---|---|---|
| Buffer | Measurement item | | Initial value | 10 days | 20 days | 30 days |
| Malic | Property | | dark reddish-brown colloid | ← | ← | ← |
| acid | | | solution, showed Tyndall | | | |
| | | | phenomenon with | | | |
| | | | transmitted light | | | |
| | pH value | | 5.32 | 5.11 | 5.01 | 4.82 |
| | Insoluble | | clear, no insoluble | clear, no | clear, no | clear, no |
| | contaminant | | contaminant | insoluble | insoluble | insoluble |
| | inspection | | | contaminant | contaminant | contaminant |
| | Content | Fe | 98.6 | — | — | 97.7 |
| | (%) | Zn | 99.5 | — | — | 98.5 |
| | | Cu | 101.3 | — | — | 101.3 |
| | | I | 99.7 | — | — | 99.0 |
| Critic | Property | | dark reddish-brown colloid | ← | ← | ← |
| acid | | | solution, showed Tyndall | | | |
| | | | phenomenon with | | | |
| | | | transmitted light | | | |
| | pH value | | 5.41 | 5.27 | 5.14 | 4.94 |
| | Insoluble | | clear, no insoluble | clear, no | clear, no | clear, no |
| | contaminant | | contaminant | insoluble | insoluble | insoluble |
| | inspection | | | contaminant | contaminant | contaminant |
| | Content | Fe | 99.1 | — | — | 98.1 |
| | (%) | Zn | 100.0 | — | — | 100.1 |
| | | Cu | 101.6 | — | — | 101.0 |
| | | I | 99.7 | — | — | 97.9 |

(Note 1)
The item of property is based on n = 1 (where “n” is the number of samples); the items of pH and content show the mean at the time of n = 3; and the item of insoluble contaminant inspection shows the observation results when n = 3.
(Note 2)
Content was measured by: atomic absorption spectrometry for Fe with ferric chloride, Zn with zinc sulfate, and Cu with copper sulfate; and the HPLC method for I with potassium iodide

As shown in Table 3, the decline of the pH for each of the samples was mild. On the other hand, for insoluble contaminant inspection, no insoluble contaminant was recognized, and for property and content, almost no change was recognized compared to the initial values.

Each of the samples to which malic acid or citric acid was added as a buffer was stable to extreme temperature as well.

### Example 3

An aqueous ferric chloride solution obtained by dissolving 94.6 g of ferric chloride into 390 mL of purified water were fed into a solution of shark chondroitin sulfate obtained by dissolving 97.74 g of shark-cartilage-derived sodium chondroitin sulfate (molecular weight of about 10,000) into 1480 mL of purified water, in a 5-fold amount of the shark-cartilage-derived sodium chondroitin sulfate to the amount of iron (Fe) in weight ratio, with stirring, while adding an amount of an aqueous sodium hydroxide solution necessary to maintain the pH at about 6.5, followed by purified water to adjust to a volume of 5000 mL. Subsequently, the resultant mixture was heated at 110° C. for 20 minutes to prepare chondroitin iron sulfate colloid solution with 4 mg/mL of iron concentration.

3.450 g of zinc sulfate was dissolved in 40 mL of purified water to prepare a zinc sulfate solution. In addition, 0.0396 g of manganese chloride, 0.2496 g of copper sulfate, 0.0129 g of selenious acid, and 0.0332 g of potassium iodide were each dissolved in 10 mL of purified water to prepare respective solutions.

To 100 mL of the chondroitin iron sulfate colloid solution, an appropriate amount of purified water was added, and a pH buffer was added. After adjusting the pH to about 5.8, the manganese chloride solution, the zinc sulfate solution, the copper sulfate solution, and the selenious acid solution were added in order, and the pH value was adjusted to 5.8 by adding an aqueous sodium hydroxide solution (1 wt/vol %). Further, the potassium iodide solution was added, and purified water was added to obtain a solution of 400 mL in total volume.

The solution was filtered using a membrane filter with a pore size of 0.22 μm, and 2 mL of the filtrate was filled into each of 10 barium-free colorless ampoules, followed by melt sealing. Subsequently, the ampoules were subjected to a high-pressure steam sterilization to prepare aqueous preparations.

As samples, three kinds of aqueous preparations each containing six elements (iron, manganese, zinc, copper, selenium and iodine) with 0.1 wt/vol % of glycine, and 0.2 wt/vol % of citric acid and sodium glutamate were prepared.

For these samples, a mixing test was conducted after mixing 2 mL of the solution to the following high-calorie intravenous nutrition infusions.

For the mixing test, observation was performed with respect to the properties, the pH, and the insoluble contaminant at room temperature just after mixing and 24 hours after mixing. The results of this test are shown in Table 4. The commercially available infusions used were “AMINOTRIPA No.2” (trade name, Otsuka Pharmaceutical Co., Ltd.) and “PNTWIN-3” (trade name, Ajinomoto Pharma Co., Ltd.).

TABLE 4

| | | | Before | Just after | 24 hours after |
|---|---|---|---|---|---|
| Infusion | Aqueous preparation | Test item | mixing | mixing | mixing |
| AMINO-TRIPA | Containing glycine | Property | Clear and | Clear yellowish | Clear yellowish |
| No. 2 | | | colorless | brown | brown |
| | | pH value | 5.58 | 5.58 | 5.51 |
| | | Insoluble | None | None | None |
| | | contaminant | | | |
| | Containing citric | Property | Clear and | Clear yellowish | Clear yellowish |
| | acid | | colorless | brown | brown |
| | | pH value | 5.59 | 5.59 | 5.51 |
| | | Insoluble | None | None | None |
| | | contaminant | | | |
| | Containing L-sodium | Property | Clear and | Clear yellowish | Clear yellowish |
| | glutamate | | colorless | brown | brown |
| | | pH value | 5.60 | 5.59 | 5.52 |
| | | Insoluble | None | None | None |
| | | contaminant | | | |
| PNTWIN-3 | Containing glycine | Property | Clear and | Clear yellowish | Clear yellowish |
| | | | colorless | brown | brown |
| | | pH value | 5.16 | 5.18 | 5.14 |
| | | Insoluble | None | None | None |
| | | contaminant | | | |
| | Containing citric | Property | Clear and | Clear yellowish | Clear yellowish |
| | acid | | colorless | brown | brown |
| | | pH value | 5.18 | 5.16 | 5.15 |
| | | Insoluble | None | None | None |
| | | contaminant | | | |
| | Containing L-sodium | Property | Clear and | Clear yellowish | Clear yellowish |
| | glutamate | | colorless | brown | brown |
| | | pH value | 5.18 | 5.18 | 5.15 |
| | | Insoluble | None | None | None |
| | | contaminant | | | |

As shown in Table 4, each of the aqueous preparations of the present invention (Example 3) did not show any change in its properties up to 24 hours after mixing in the commercially available infusions, and precipitates such as insoluble contaminants were not observed. In addition, almost no change in pH was recognized.

### Example 4

Similar to the Examples above, an aqueous ferric chloride solution obtained by dissolving 94.6 g of ferric chloride into 390 mL of purified water were fed into a solution of shark chondroitin sulfate obtained by dissolving 97.74 g of shark-cartilage-derived sodium chondroitin sulfate (molecular weight of about 10,000) into 1480 mL of purified water, in a 5-fold amount of the shark-cartilage-derived sodium chondroitin sulfate to the amount of iron (Fe) in weight ratio, with stirring, while adding an amount of an aqueous sodium hydroxide solution necessary to maintain the pH at about 6.5, followed by purified water to adjust to a volume of 5000 mL. Subsequently, the resultant mixture was heated at 110° C. for 20 minutes to prepare chondroitin iron sulfate colloid solution with 4 mg/mL of iron concentration. Subsequently, heat treatment was carried out to prepare and chondroitin iron sulfate colloid solution with 4 mg/mL of iron concentration.

172.50 g of zinc sulfate was dissolved in 2,000 mL of purified water to prepare a zinc sulfate solution. In addition, 12.48 g of copper sulfate and 1.66 g of potassium iodide were each dissolved in 500 mL of purified water to prepare respective solutions.

To 5,000 mL of the chondroitin iron sulfate colloid solution, an appropriate amount of purified water was added. After adjusting the pH to about 6, the zinc sulfate solution, and the copper sulfate solution were added in order, and the pH was adjusted to 5.8 by adding an aqueous sodium hydroxide solution (1 wt/vol %). Further, the potassium iodide solution was added and purified water was added to obtain a solution of 20,000 mL in total volume.

The solution was filtered using a membrane filter with a pore size of 0.22 μm, and 2 mL of the filtrate was filled into each of 445 barium-free colorless ampoules, followed by melt sealing. Subsequently, the ampoules were subjected to a high-pressure steam sterilization to prepare aqueous preparations.

The prepared preparations were confirmed to be, by conducting stability evaluation for 6 months at 40° C., stable with respect to the property, the pH, insoluble contaminant inspection, insoluble fine particle test, and content.

Effect of the Invention

The aqueous preparation of the present invention can be safely applied in clinical use without worry of BSE infection. It is pharmaceutically stable, and is stable in an infusion such as a high-calorie intravenous nutrition infusion.

What is claimed is:

1. A stabilized aqueous preparation comprising a shark-derived chondroitin iron sulfate colloid and a trace element necessary for the human body.

2. An aqueous preparation according to claim 1, further comprising a pH buffer.

3. An aqueous preparation according to claim 1, wherein the shark-derived chondroitin iron sulfate colloid is produced from a shark-derived chondroitin sulfate.

4. An aqueous preparation according to claim 2, wherein the shark-derived chondroitin iron sulfate colloid is produced from a shark-derived chondroitin sulfate.

5. An aqueous preparation according to claim 3, wherein the shark-derived chondroitin sulfate is an alkali metal salt of the shark-derived chondroitin sulfate.

6. An aqueous preparation according to claim 4, wherein the shark-derived chondroitin sulfate is an alkali metal salt of the shark-derived chondroitin sulfate.

7. An aqueous preparation according to claim 3, wherein the shark-derived chondroitin sulfate is shark-derived sodium chondroitin sulfate.

8. An aqueous preparation according to claim 4, wherein the shark-derived chondroitin sulfate is shark-derived sodium chondroitin sulfate.

9. An aqueous preparation according to claim 7, wherein the shark-derived sodium chondroitin sulfate has an average molecular weight of from about 10,000 to about 25,000; a limiting viscosity of from about 0.27 to about 0.65 dl/g; and a sulfur content of from about 6.4 to about 7.0 wt/wt %.

10. An aqueous preparation according to claim 8, wherein the 5 shark-derived sodium chondroitin sulfate has an average molecular weight of from about 10,000 to about 25,000; a limiting viscosity of from about 0.27 to about 0.65 dl/g; and a sulfur content of from about 6.4 to about 7.0 wt/wt %.

11. An aqueous preparation according to claim 2, wherein the pH buffer is one or more compounds selected from the group consisting of glycine, L-valine, L-arginine, L-histidine, acetic acid, malonic acid, maleic acid, succinic acid, malic acid, tartaric acid, citric acid, L-sodium glutamate, disodium phthalate, and disodium fumarate.

12. An aqueous preparation according to claim 11, wherein the pH buffer is glycine, malic acid, citric acid or L-sodium glutamate.

13. An aqueous preparation according to claim 11, wherein the trace element is one or more elements selected from the group consisting of selenium, zinc, copper, iodine, and manganese.

14. An aqueous preparation according to claim 13, wherein trace elements are zinc, copper, and iodine.

15. An aqueous preparation according to claim 13, wherein trace elements are zinc, copper, iodine, and manganese.

16. An aqueous preparation according to claim 13, wherein trace elements are selenium, zinc, copper, iodine, and manganese.

17. An aqueous preparation according to claim 1, wherein a weight ratio of iron:the shark-derived sodium chondroitin iron sulfate colloid is from about 1:4 to about 1:6.

18. An aqueous preparation according to claim 2, wherein a weight ratio of iron:the shark-derived sodium chondroitin iron sulfate colloid is from about 1:4 to about 1:6.

19. An aqueous preparation according to claim 17, wherein the weight ratio of the iron:the shark-derived chondroitin iron sulfate colloid is about 1:5.

20. An aqueous preparation according to claim 18, wherein the weight ratio of the iron:the shark-derived chondroitin iron sulfate colloid is about 1:5.

21. An aqueous preparation according to claim 2, wherein the concentration of the pH buffer is from about 0.01 to about 1.0 wt/vol %.

22. An aqueous preparation according to claim 1, wherein the preparation contains the shark-derived chondroitin iron sulfate colloid produced by adding an aqueous ferric salt solution and an aqueous alkali metal hydroxide solution to an aqueous shark-derived chondroitin sulfate solution so that a pH of the resulting mixture is adjusted to a pH within a range of from about 5.5 to about 7.5.

23. A method for manufacturing an aqueous preparation containing a shark-derived chondroitin iron sulfate colloid, comprising: adding an aqueous ferric salt solution and an aqueous alkali metal hydroxide solution to an aqueous shark-derived chondroitin sulfate solution so that a pH of the resulting mixture is adjusted to a pH within a range of from about 5.5 to about 7.5; heating the resulting mixture; and adding a pH buffer and a trace element.

24. A method for manufacturing an aqueous preparation containing a shark-derived chondroitin iron sulfate colloid according to claim 23, wherein the shark-derived chondroitin sulfate is shark-derived sodium chondroitin sulfate, and the alkali metal hydroxide is sodium hydroxide.
